(19) United States
(12) Patent Application Publication
(10) Pub. No.: US 2026/0310688 A1
(43) Pub. Date: Oct. 8, 2026

(54) SYSTEM AND METHOD FOR REAL-TIME MONITORING UTILIZING MACHINE-LEARNING BASED UPON SENSOR DATA

(71) Applicant: State Farm Mutual Automobile Insurance Company, Bloomington, IL (US)

(72) Inventors: Edward W. Breitweiser, Bloomington, IL (US); Julia Marsaglia, Normal, IL (US); Eric R. Moore, Heyworth, IL (US); Andrew T. Swartz, Mesa, AZ (US); Harrison Lin, Normal, IL (US); Eric A. Finley, Bloomington, IL (US); Jennifer L. Crawford, Normal, IL (US)

(73) Assignee: State Farm Mutual Automobile Insurance Company, Bloomington, IL (US)

(21) Appl. No.: 19/194,785

(22) Filed: Apr. 30, 2025

Related U.S. Application Data
(60) Provisional application No. 63/785,204, filed on Apr. 8, 2025.

(51) Int. Cl. G06F 16/28

(52) U.S. Cl. CPC G06F 16/287

(57) ABSTRACT

System and methods for analyzing data to generate personalized response(s) are disclosed. The method may include, such as by one or more processors, transceivers, and/or sensors: (1) receiving collected sensor data from external data stores; (2) utilizing an NLP model and a computer vision model to categorize the collected data into improvement category, positive category, or neutral category; (3) retrieving policy data corresponding to the user from data store(s); (4) inputting the categorized data and the policy data into a trained machine-learning model to generate personalized responses; (5) generating a presentation of the personalized responses in a device; (6) receiving updated sensor data from the sensor(s) associated with the user; (7) analyzing the updated sensor data to determine a compliance level for the categories to the personalized response(s); (8) comparing the compliance level to a category threshold for the categories; and/or (9) outputting an updated presentation based upon the comparing.

### CROSS-REFERENCE TO RELATED APPLICATION

This patent application claims the benefit of priority to U.S. Provisional Application No. 63/785,204, filed on Apr. 8, 2025, the entirety of which is incorporated herein by reference.

### TECHNICAL FIELD

This present disclosure relates generally to the field of data processing and data-driven recommendation systems. In particular, the present disclosure relates to processing data associated with users for dynamically generating personalized responses using machine-learning techniques.

### BACKGROUND

Conventional approaches to delivering recommendations may often rely upon static rule-based systems, generic notification frameworks, or siloed data sources, which may lack the technical ability to process multimodal user data, such as audio, visual, environmental, and/or behavioral data. These systems may typically fail to categorize user data contextually or adapt recommendations based upon longitudinal trends in the user's physical and/or mental health indicators. Furthermore, traditional systems may not leverage dynamic feedback loops that incorporate the user's improvement or deterioration over time to refine future recommendations, resulting in stale, non-personalized outputs.

Traditional methods may also be limited in scalability and personalization, as these methods may not utilize machine-learning models capable of continuously learning from categorized user data and behavioral patterns. Additionally, conventional methods may often lack cross-domain integration, such as environmental factors, social interaction metrics, and/or cost-saving considerations, leading to fragmented, non-contextualized user support. This may result in reduced system responsiveness, non-adaptive outputs, limited scalability, and an inability to provide timely, actionable, and personalized guidance based upon real-time or evolving user data. Conventional techniques may include other ineffectiveness, encumbrances, inefficiencies, and drawbacks, as well.

### SUMMARY

The present embodiments may relate, inter alia, to dynamic personalization and optimization of recommendations through the integration of multimodal user data and iterative machine learning refinement, such as those discussed above and elsewhere herein. Specifically, the present computer systems and computer-implemented methods may solve technical challenges by implementing a feedback loop that continuously assesses the progression of user behavior and wellness indicators. The user data may be categorized into relevant categories using advanced natural language processing (NLP) and computer vision models, while also leveraging real-time assessment to retrain machine-learning models. Such retraining may allow the system to dynamically adjust future recommendations based upon real-time data, ensuring that the provided guidance is contextually relevant, personalized, and adaptable to the user's changing physical, mental, and environmental conditions.

In one aspect, a computer-implemented method for analyzing data to generate one or more personalized responses may be provided. The computer-implemented method may be implemented via one or more local or remote processors, servers, transceivers, memory units, mobile devices, voice bots or chatbots, ChatGPT bots, InstructGPT bots, Codex bots, Google Bard bots, and/or other electronic or electrical components, which may be in wired or wireless communication with one another and operate as input and/or output devices. In one instance, the computer-implemented method may be performed by one or more local or remote processors of a computing system in communication with one or more local or remote data sources.

The computer-implemented method may include, via one or more local or remote processors, transceivers, sensors, servers, and/or other components: (1) receiving, by the one or more processors, collected sensor data from one or more external data stores, wherein the collected sensor data corresponds to one or more sensors associated with at least one user; (2) utilizing, by the one or more processors, an NLP model and a computer vision model to categorize the collected sensor data into one or more categories, wherein the one or more categories include at least one of (i) an improvement category, (ii) a positive category, or (iii) a neutral category; (3) retrieving, by the one or more processors, policy data corresponding to the at least one user from one or more data stores; (4) inputting, by the one or more processors, the categorized data and the policy data into a trained machine-learning model to generate the one or more personalized responses (such as visual, graphical, textual, audible, and/or verbal responses) for the at least one user; (5) generating, by the one or more processors, a presentation of the one or more personalized responses in a user interface of at least one device associated with the at least one user; (6) receiving, by the one or more processors, updated sensor data from the one or more sensors associated with the at least one user; (7) analyzing, by the one or more processors, the updated sensor data to determine a compliance level for at least one of the one or more categories to the one or more personalized responses; (8) comparing, by the one or more processors, the compliance level to a category threshold corresponding to the one or more categories; and/or (9) outputting, by the one or more processors, an updated presentation (such as visual, graphical, textual, audible, and/or verbal presentations, such as presentations presented via a voice bot and/or display screen, or other input/output device) based upon the comparing in the user interface of the at least one device. The method may include additional, less, or alternate functionality, including that discussed elsewhere herein.

In another aspect, a computer system for analyzing data to generate one or more personalized responses may be provided. The computer system may be implemented via one or more local or remote processors, servers, transceivers, memory units, mobile devices, voice bots or chatbots, ChatGPT bots, InstructGPT bots, Codex bots, Google Bard bots, and/or other electronic or electrical components, which may be in wired or wireless communication with one another. In one instance, the computer system may be performed by one or more local or remote processors of a computing system in communication with one or more local or remote data sources, and at least one non-transitory computer readable medium storing instructions which, when executed by the one or more processors, cause the one or more processors to perform certain operations.

The computer system may include, via one or more processors, non-transitory computer readable medium, transceivers, sensors, servers, and/or other components to perform operations that may include: (1) receiving collected sensor data from one or more external data stores, wherein the collected sensor data corresponds to one or more sensors associated with at least one user; (2) utilizing an NLP model and a computer vision model to categorize the collected sensor data into one or more categories, wherein the one or more categories include at least one of (i) an improvement category, (ii) a positive category, or (iii) a neutral category; (3) retrieving policy data corresponding to the at least one user from one or more data stores; (4) inputting the categorized data and the policy data into a trained machine-learning model to generate the one or more personalized responses for the at least one user; (5) generating a presentation (such as a visual, graphical, textual, audible, and/or verbal presentation via a voice bot, display screen, or other input/output device) of the one or more personalized responses in a user interface of at least one device associated with the at least one user; (6) receiving updated sensor data from the one or more sensors associated with the at least one user; (7) analyzing the updated sensor data to determine a compliance level for at least one of the one or more categories to the one or more personalized responses; (8) comparing the compliance level to a category threshold corresponding to the one or more categories; and/or (9) outputting an updated presentation (such as a visual, graphical, textual, audible, and/or verbal presentation) based upon the comparing in the user interface of the at least one device. The system may include additional, less, or alternate functionality, including that discussed elsewhere herein.

In yet another aspect, a non-transitory computer readable medium for analyzing data to generate one or more personalized responses may be provided. The non-transitory computer readable medium may be implemented via one or more local or remote processors, servers, transceivers, memory units, mobile devices, voice bots or chatbots, ChatGPT bots, InstructGPT bots, Codex bots, Google Bard bots, and/or other electronic or electrical components, which may be in wired or wireless communication with one another. In one instance, the non-transitory computer readable medium may be performed by one or more local or remote processors of a computing system in communication with one or more local or remote data sources, and at least one non-transitory computer readable medium storing instructions which, when executed by the one or more processors, cause the one or more processors to perform certain operations.

The non-transitory computer readable medium may include, via one or more processors, transceivers, sensors, servers, and/or other components: (1) receiving collected sensor data from one or more external data stores, wherein the collected sensor data corresponds to one or more sensors associated with at least one user; (2) utilizing an NLP model and a computer vision model to categorize the collected sensor data into one or more categories, wherein the one or more categories include at least one of (i) an improvement category, (ii) a positive category, or (iii) a neutral category; (3) retrieving policy data corresponding to the at least one user from one or more data stores; (4) inputting the categorized data and the policy data into a trained machine-learning model to generate the one or more personalized responses for the at least one user; (5) generating a presentation (such as a visual, graphical, textual, audible, and/or verbal presentation) of the one or more personalized responses in a user interface of at least one device associated with the at least one user; (6) receiving updated sensor data from the one or more sensors associated with the at least one user; (7) analyzing the updated sensor data to determine a compliance level for at least one of the one or more categories to the one or more personalized responses; (8) comparing the compliance level to a category threshold corresponding to the one or more categories; and/or (9) outputting an updated presentation (such as a visual, graphical, textual, audible, and/or verbal presentation) based upon the comparing in the user interface of the at least one device. The operations may include additional, less, or alternate functionality, including that discussed elsewhere herein.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 depicts a diagram showing an exemplary architecture for processing user data, categorizing the processed data into one or more categories, and/or generating personalized recommendations based upon machine learning analysis, according to certain aspects of the disclosure.

FIG. 2A is an exemplary flowchart of a computer-implemented or computer-based process for analyzing data to generate one or more personalized responses, according to one or more embodiments.

FIG. 2B is an exemplary diagram that illustrates a scenario of a user interacting with a multi-sensor environment to receive personalized recommendations, according to one or more embodiments.

FIG. 3 shows an exemplary machine-learning training flow chart, according to one or more embodiments.

FIG. 4 illustrates an implementation of an exemplary computer system that executes one or more techniques presented herein.

Advantages will become more apparent to those skilled in the art from the following description of the preferred embodiments that have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

The present embodiments may relate, inter alia, to computer systems and computer-implemented methods that may solve technical challenges by utilizing advanced machine-learning models and real-time data processing techniques to dynamically categorize and analyze multimodal user data, including behavioral, environmental, and sensory data. The computer systems and computer-implemented methods may employ natural language processing (NLP) and computer vision algorithms to categorize user data into actionable insights, which may then be continuously refined through feedback loops that assess improvements or deteriorations in user well-being. By incorporating contextual awareness from diverse data sources and integrating adaptive learning mechanisms, the computer systems and computer-implemented methods may autonomously update and personalize recommendations based upon real-time user behaviors, improving user engagement and adherence.

Conventional systems for providing wellness and behavioral recommendations may be technically constrained by a reliance upon static, rule-based frameworks and pre-defined heuristics. These systems may lack the flexibility to process complex, heterogeneous data sources. As a result, these systems may not dynamically adapt to variations in user behavior, environmental conditions, and/or personal wellness metrics. Such systems may treat all users uniformly, without accounting for unique behavioral patterns, cultural factors, or evolving mental and physical health indicators. The absence of real-time data integration and adaptive learning mechanisms may limit these conventional systems to generic, one-size-fits-all notifications, which often leads to user fatigue and reduced engagement over time.

Traditional methods may be technically challenged to process and categorize unstructured or multimodal data. For example, these methods may lack the capability to analyze audio cues indicative of physical health conditions (e.g., coughing or wheezing), visual data relating to user activity, or contextual environmental factors like air quality or social interaction levels. Even when data from multiple domains are available, conventional methods often operate in isolated silos, preventing meaningful cross-domain analysis. This fragmented approach may result in technically incomplete user profiles, leading to inaccurate, irrelevant, or untimely recommendations that may fail to address the root cause of a user's behavioral or wellness issues.

Conventional approaches may face scalability and/or personalization challenges, particularly in large-scale deployment across diverse user populations. Without machine-learning based personalization, these approaches cannot handle variations in user preferences, demographics, behavioral tendencies, or environmental contexts. These approaches may also be technically limited in the ability to automatically recalibrate the recommendation logic based upon user-specific improvements or deterioration assessments. As a result, such approaches may be prone to becoming obsolete or inefficient without manual intervention, periodic reprogramming, or costly human oversight. The absence of adaptive learning mechanisms, coupled with the inability to process and correlate diverse, real-time data inputs, may render conventional approaches technically inept at delivering accurate, personalized, and/or contextually relevant recommendations.

### Exemplary Computer System

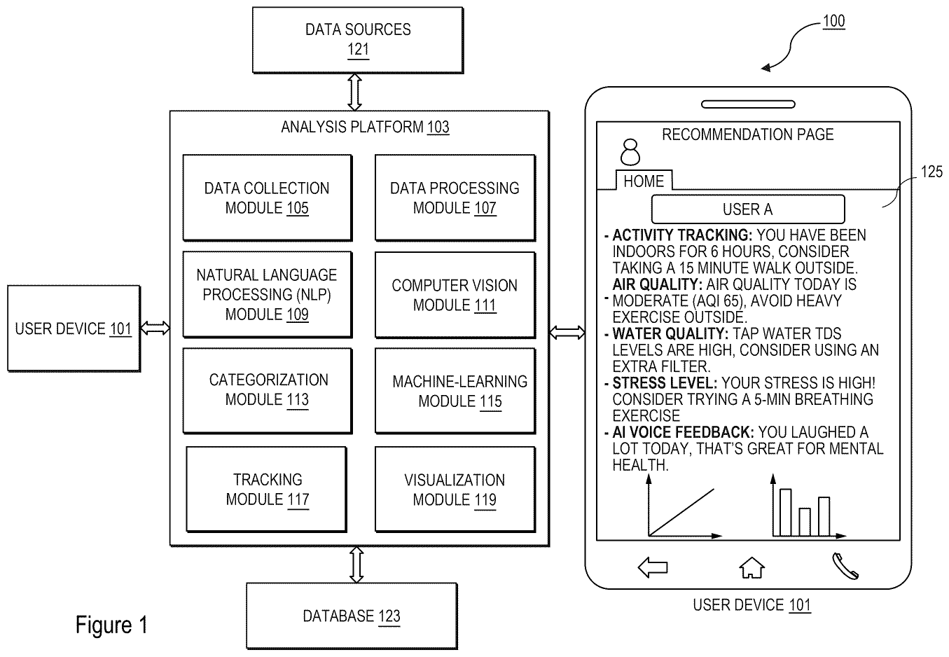

To address technical challenges, such as the above, system 100 of FIG. 1 may improve upon the conventional systems by integrating a dynamic, machine-learning driven approach that may continuously adapt to user behavior and evolving needs. Unlike static rule-based systems, the system 100 may leverage a trained machine-learning model that may process multimodal data sources, ranging from environmental factors, such as air and water quality, to physical health indicators like activity levels and audio signals indicative of health conditions (e.g., coughing or wheezing). The system 100 may categorize and analyze this data in real-time, and may generate highly personalized recommendations that are not only contextually relevant, but also tailored to the specific circumstances of each user. By incorporating these diverse data points, the system 100 may provide a comprehensive view of user health, addressing both physical and mental wellness in a holistic manner. This holistic approach may ensure that the system 100 provides accurate, actionable, and contextually relevant recommendations, thereby optimizing user engagement and health outcomes over time.

Unlike conventional systems that typically rely upon simple data input and static rules, the system 100 may utilize advanced technologies, such as NLP and computer vision, to process and interpret complex, unstructured data. In one instance, NLP may be utilized to analyze and classify text-based inputs (e.g., voice commands, written feedback, or health-related messages), allowing the system 100 to understand nuanced expressions of physical and mental well-being. In one instance, computer vision techniques may enable the system 100 to analyze visual inputs, such as user activity or posture, through cameras or wearable devices, providing insights into physical health or social engagement. These technologies may allow the system 100 to capture and interpret a much broader range of user data, including subtle physical and emotional signals that the traditional systems may find technically challenging to process. By combining NLP, computer vision, and other advanced methods, the system 100 may provide a richer, multi-dimensional analysis of users' wellness, enabling the system 100 to generate more personalized, context-sensitive recommendations.

Additionally, the system 100 may utilize a feedback mechanism, where recommendations may be dynamically adjusted based upon an ongoing assessment of user progress. The system 100 may track adherence by the users to the provided guidance and may analyze the users' actions and feedback to identify trends in health and behavioral patterns. When the system 100 detects improvements or deteriorations in the categories (e.g., physical health, stress levels, social engagements), the system 100 may feed the data to the machine-learning model for retraining and fine-tuning for future recommendations. This adaptive learning mechanism may ensure that the system 100 remains responsive and evolves alongside the user's needs, providing more accurate and effective interventions as users progress through the wellness journey.

Furthermore, the system 100 may incorporate cross-domain integration, processing, and correlating data from various categories, including mental health categories, physical well-being categories, environmental conditions, and/or cost-saving behaviors. This multidimensional approach may improve the accuracy and relevance of recommendations and may also allow the system 100 to generate insights across different aspects of a user's life, fostering comprehensive wellness support. By combining real-time data processing with adaptive machine-learning, the system 100 may overcome the scalability and personalization limitations of conventional systems, delivering context-aware and individualized recommendations.

FIG. 1 depicts a diagram showing an exemplary architecture for processing user data, categorizing the processed data into one or more categories, and generating personalized recommendations based upon machine-learning analysis, according to certain aspects of the disclosure. FIG. 1 may include the computing system 100 that may include a user device 101, an analysis platform 103, data sources 121, and/or a database 123. It should be understood that other implementations of computing system 100 may omit one or more of the foregoing components and/or may include additional components, as the case may be.

In one instance, the user device 101 may include a smartphone, a tablet, a wearable device, or any portable electronic device capable of communicating with external systems. The user device 101 may be operatively coupled to one or more sensors configured to monitor various physical, environmental, and/or behavioral parameters associated with the user. The one or more sensors may include, but are not limited to, motion sensors (e.g., gyroscope, accelerometers), location sensors (e.g., GPS), biometric sensors (e.g., heart rate monitors, body temperature sensors, galvanic skin response sensors), audio sensors (e.g., microphones), and/or environmental sensors (e.g., air quality sensors, light sensors). In some instances, the user device 101 may further be connected to external wearable devices, such as smartwatches, smart glasses, or smart clothing, each comprising embedded sensors configured to monitor user activity, physiological conditions, and/or environmental factors. The collected sensor data may include activity levels, location information, biometric reading, environmental quality metrics, and/or audio data, which is transmitted to the analysis platform 103 for processing, categorization, and generation of personalized recommendations based upon the user's physical and mental well-being.

In one instance, the analysis platform 103 may include a platform with multiple interconnected components. The analysis platform 103 may include one or more servers, intelligent networking devices, computing devices, components, and/or corresponding software for collecting data associated with one or more users, the data comprising physical, environmental, behavioral, and/or contextual information captured by the user device 101 and the one or more sensors. Upon collecting the data, the analysis platform 103 may utilize NLP and computer vision models to analyze the data and categorize the collected data into one or more categories, such as a positive category, an improvement category, and/or a neutral category, based upon predefined or dynamically updated criteria related to user well-being.

The analysis platform 103 may input the categorized data into a trained machine-learning model that processes the categorized data to generate one or more personalized recommendations for the user. The recommendations may relate to improving physical health, enhancing mental well-being, fostering community engagement, or promoting cost-saving behaviors. The analysis platform 103 may generate a user interface presentation displaying the personalized recommendations and may continuously track one or more actions performed by the user to determine adherence to the provided recommendations. Additionally, the analysis platform 103 may evaluate the tracked actions and the progression of categorized data to assess whether there is an improvement or deterioration in the user's categorized well-being indicators. Based upon this assessment, the analysis platform 103 may input the assessment results into the trained machine-learning model for retraining to refine the model to enhance relevance, accuracy, and personalization of future recommendations.

In one instance, the analysis platform 103 may include a data collection module 105, a data processing module 107, an NLP module 109, a computer vision module 111, a categorization module 113, a machine-learning module 115, a tracking module 117, and a visualization module 119, or any combination thereof. As used herein, terms such as “component” or “module” generally encompass hardware and/or software, e.g., that a processor or the like used to implement associated functionality. It is contemplated that the functions of these components are combined in one or more components or performed by other components of equivalent functionality.

In one instance, the data collection module 105 may include various software applications (e.g., data mining applications in Extended Meta Language (XML)) that automatically search for and return the relevant data. The data collection module 105 may be communicatively coupled to the user device 101, one or more wearable devices, and/or external data sources (e.g., data sources 121) to receive real-time and/or historical data associated with one or more users. The data collected by the data collection module 105 may include, but is not limited to, sensor data (e.g., activity levels, biometric readings, environmental conditions), user behavioral data (e.g., application usage patterns, online activity, physical location), audio data (e.g., speech patterns, coughing sounds), and/or external contextual data (e.g., weather conditions, air quality indices, water quality reports). The data collection module 105 may also receive user-provided inputs, such as preferences, dietary habits, or self-reported stress levels. The data collection module 105 may automate data extraction, ensuring continuous updates and validation to maintain data integrity. The data may be collected and processed by the system 100 if the users have consented to the use of their data for processing to generate recommendations.

In one instance, the data processing module 107 may organize, filter, and prepare the collected user data for further analysis. The data processing module 107 may receive raw data from the data collection module 105, which may include sensor data, user behavioral data, environmental data, and/or contextual information. The data processing module 107 may employ advanced algorithms to clean and preprocess the data, ensuring that the data is in a suitable format for analysis. In one example, data cleansing may include removing or correcting erroneous data (e.g., redundant, incomplete, or incorrect data) to create high-quality data. The data cleansing technique may also include a data enhancement technique, where data may be made more complete by adding related information. In another example, the data preprocessing techniques may include filtering out noise, handling missing values, and/or aggregating data to provide a comprehensive overview. Additionally, the data processing module 107 may normalize the data into a consistent format and structure suitable for downstream categorization and analysis.

In one instance, the NLP module 109 may analyze and interpret textual and/or audio-based inputs associated with one or more users. The NLP module 109 may be operatively connected to the data processing pipeline and may process unstructured data, such as user-generated text, speech transcripts, and/or written inputs, to extract meaningful information relevant to the user's physical health, mental well-being, and/or behavioral patterns. The NLP module 109 may perform various functions including tokenization, entity recognition, sentiment analysis, keyword extraction, and/or context categorization. The NLP module 109 may further analyze linguistic patterns, emotional tone, and/or frequencies of specific keywords to identify indicators of positive, improvement, or neutral behavior.

In one example, the NLP module 109 may analyze messages, social media posts, journal entries, and/or speech inputs captured by the user device 101 to detect expressions of stress, loneliness, and/or positive engagement. In one instance, the NLP module 109 may identify phrases indicative of stress, such as “I feel overwhelmed” or “I can't handle this,” and may determine that the user is highly stressed. In another instance, the NLP module 109 may detect positive language, such as “had a great walk today” or “feeling calm after mediation,” and may determine that the user is relaxed. Additionally, the NLP module 109 may process voice data captured by the user device 101 to detect changes in speech tone, pace, and/or language usage that may suggest mental health concerns. For example, slower speech, monotone delivery, and/or increased pauses may indicate symptoms of fatigue. Conversely, rapid speech and/or elevated pitch may correlate with anxiety or heightened stress levels. The NLP module 109 may also analyze the frequency and choice of negative words (e.g., stressed, lonely, tired, sad, etc.) or positive words (e.g., happy, relaxed, grateful, excited, calm, loved, etc.) used during conversations to assess the user's emotional state.

In one instance, the computer vision module 111 may analyze images and/or videos associated with one or more users to extract visual indicators relevant to physical health, mental well-being, and/or behavioral patterns. The computer vision module 111 may be operatively connected to the data processing pipeline and may be designed to process visual data captured by one or more user devices 101 and/or other wearable devices equipped with cameras. The computer vision module 111 may perform various functions, such as object detection, facial expression recognition, posture analysis, activity recognition, and/or environmental context identification. The computer vision module 111 may further process visual data to identify user behaviors, detect physical conditions, and/or assess environmental factors, such as exposure to nature or time spent indoors.

In one example, the computer vision module 111 may analyze image and/or video streams to detect whether the user is located indoors or outdoors, based upon lighting conditions, visible natural elements, and/or environmental markers. In another example, the computer vision module 111 may analyze facial expressions of the user to detect emotional indicators, such as smiling, frowning, and/or signs of fatigue which may correlate to mental well-being. The computer vision module 111 may also monitor body posture and/or movement patterns to identify sedentary behavior, physical activity levels, or potential signs of physical discomfort. Additionally, the computer vision module 111 may assess images and/or videos to assess dietary choices, detect the presence of other individuals in the environment to infer social engagement, and/or evaluate the user's workplace setup to recommend ergonomic improvements.

In one instance, the categorization module 113 may categorize processed data into predefined categories, such as a positive category, an improvement category, or a neutral category, based upon extracted features. The categorization module 113 may receive processed data from the data processing module 107, the NLP module 109, and/or the computer vision module 111, and may apply rule-based logic, statistical analysis, and/or predefined categorization models to determine the appropriate category. The categorization module 113 may evaluate multiple factors such as sentiment, detected activities, environmental contexts, and/or behavioral patterns to assign a categorization label to each data point. In one example, if the categorization module 113 detects frequent outdoor activities, positive language in user messages, and/or a consistent sleep schedule, the data may be categorized as positive, indicating well-being. Conversely, if the categorization module 113 detects prolonged indoor time, a lack of movement, or speech patterns associated with stress, the data may be categorized as corresponding to the improvement category, prompting intervention strategies. A neutral categorization may be assigned when user data does not strongly indicate either a positive or an improvement trend, such as routine activities or non-emotional language in text inputs. This categorization process may enable the system 100 to dynamically adjust recommendations based upon user status and behavioral changes over time.

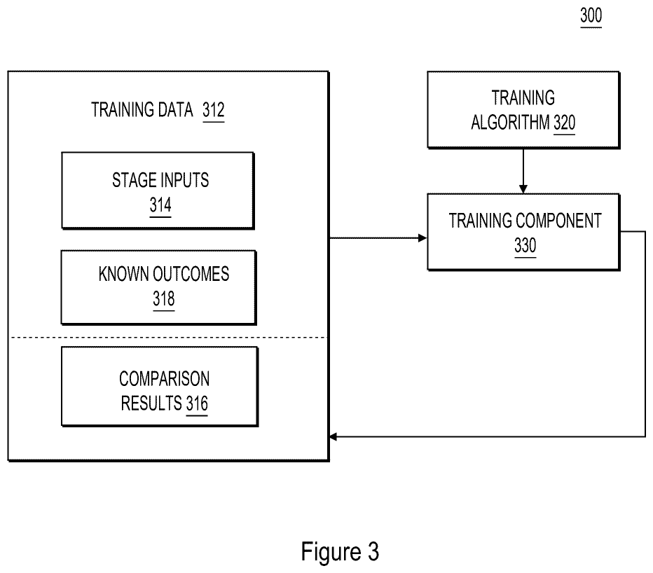

In one instance, the machine-learning module 115 may be configured for supervised machine learning, utilizing training data (e.g., training data 312 illustrated in the training flow chart of FIG. 3). The trained model may be configured for processing historical data associated with the users (e.g., activity data, behavioral data, biometric readings, environmental conditions, etc.) to learn patterns associated with the user's physical health and mental well-being. In one example, the machine-learning module 115 may perform model training using training data (e.g., data from other modules, that contain input and correct output, to allow the model to learn over time). The training may be performed based upon the deviation of a processed result from a documented result when the inputs are fed into the machine-learning model (e.g., an algorithm measures its accuracy through the loss function, adjusting until the error has been sufficiently minimized). The trained model may utilize exponential smoothing, autoregressive integrated moving average (ARIMA), or long short-term memory (LSTM) neural networks to analyze one or more features associated with users to identify behavioral patterns, detect trends in physical and mental well-being, assess deviations from expected behavior, predict potential risks, and/or generate personalized recommendations.

In one instance, the machine-learning module 115 may randomize the ordering of the training data, visualize the training data to identify relevant relationships between different variables, identify any data imbalances, and split the training data into two parts where one part may be for training a model and the other part may be for validating the trained model, de-duplicating, normalizing, correcting errors in the training data, and so on. The machine-learning module 115 may implement various machine learning techniques, e.g., neural network (e.g., recurrent neural networks, graph convolutional neural networks, deep learning neural networks), decision tree learning, random forest, association rule learning, inductive programming logic, K-nearest neighbors, cox proportional hazards model, support vector machines, Bayesian models, Gradient boosted machines (GBM), LightGBM (LGBM), Xtra tree classifier, etc. Implementation of the machine-learning module 115 is discussed in detail below.

In one instance, the machine-learning module 115 may generate personalized recommendations based upon the categorized data associated with one or more users. The machine-learning module 115 may receive input data categorized by the categorization module 113, and may process this data using one or more trained machine-learning models. The machine-learning module 115 may analyze historical data and real-time categorized data to identify patterns, correlations, and trends in user behavior, physical activity, environmental exposure, and/or emotional state. Based upon the identified patterns, the machine-learning module 115 may dynamically generate tailored recommendations aimed at promoting physical health, improving mental well-being, encouraging social engagement, and/or reducing harmful behaviors. The machine-learning module 115 may further update and refine recommendation strategies over time as additional categorized data become available, thereby continuously improving the relevance and effectiveness of the recommendations.

In one example, the machine-learning module 115 may detect a recurring improvement categorization related to prolonged indoor time and sedentary behavior, and may generate a recommendation prompting the user to engage in an outdoor activity or take a short walk. In another example, the machine-learning module 115 may identify a trend of negative sentiment in the user's text messages and audio inputs, and may recommend mindfulness exercises or stress-relief videos. In a further example, if the categorized data indicates frequent unhealthy food choices through location data and food images, the machine-learning module 115 may suggest healthier nearby restaurant options. In another example, the machine-learning module 115 may identify a lack of recent social engagement through inactivity in the user's communication patterns, and may recommend contacting friends or participating in community events. In an additional example, the machine-learning module 115 may observe an improvement trend, such as increased physical activity and positive sentiment, and may reinforce such behaviors by providing congratulatory messages or incentivized goals to maintain the positive trajectory.

In one instance, the tracking module 117 may monitor and record user actions in response to the generated recommendations. The tracking module 117 may continuously collect data related to the user activities, behavior, and/or environmental interactions following the issuance of recommendations by the machine-learning module 115. This may include detecting whether the user followed the prompt to engage in physical activity, improving dietary habits, reducing screen time, and/or initiating social interactions. The tracking module 117 may correlate the recorded user actions with the original recommendation to assess adherence and responsiveness. Additionally, the tracking module 117 may analyze temporal patterns to determine short-term and long-term behavioral changes, enabling the system 100 to measure the effectiveness of recommendations over time. The tracking module 117 may compare the collected data to predefined adherence criteria that may correspond to expected behaviors or actions associated with the recommendations. The tracking module 117 may then determine a compliance level for each of the categories to the recommendations, and may compare the compliance level to a category threshold corresponding to the categories.

In one instance, the tracking module 117 may track the progression of categorized data over a predetermined time period to determine one or more trends indicative of positive or negative changes in physical, mental, or social well-being. Based upon this analysis, the machine-learning module 115 may recalibrate the trained machine-learning models by adjusting one or more model parameters, retraining the trained machine-learning models with updated data, and/or incorporating new behavioral patterns observed in the user's data. This dynamic recalibration may allow the machine-learning models to adapt to the user's evolving needs and circumstances, ensuring that future recommendations remain relevant, effective, and personalized. For example, if a user consistently ignores certain types of recommendations, the recalibrated models may reduce the frequency of such prompts and instead focus upon alternative strategies more likely to encourage positive behavior.

In one instance, the visualization module 119 may enable a presentation of a graphical user interface (GUI) in the user device 101 that may facilitate visualization of one or more recommendations (e.g., display 125). The visualization module 119 may convert the recommendations generated by the machine-learning module 115 into user-friendly visual, aural, and/or textual prompts. The visualization module 119 may employ various application programming interfaces (APIs) or other function calls corresponding to the application upon the user device 101, thus enabling the display of graphics primitives such as graphs, edges, icons, menus, buttons, data entry fields, etc. For example, the visualization module 119 may generate the display 125 stating “you have been indoors for 6 hours, consider taking a 15-minute walk outside” based upon the prolonged inactivity detected by the system. The display 125 may also include real-time air quality information (e.g., air quality index is high in your area, it is recommended to stay indoors until conditions improve) or water quality alert (e.g., tap water quality is below recommended levels, consider filtering the water before consumption). Additionally, the display 125 may include stress level assessments. The visualization module 119 may ensure that the information is clear, timely, and personalized, enhancing the user's ability to make informed decisions about their physical and mental well-being. The visualization module 119 may also comprise a variety of interfaces, for example, interfaces for data input and output devices, referred to as I/O devices, storage devices, and the like. Still further, the visualization module 119 may be configured to operate in connection with augmented reality (AR) processing techniques, wherein various applications, graphic elements, and features interact.

In one instance, the system 100 may be configured to retrieve supplemental data from data sources 121 for additional context. The data sources 121 may include environmental databases, such as air quality monitoring services, weather databases, and/or governmental or municipal databases reporting upon water quality standards and real-time contamination alerts, providing contextual information about the user's surroundings. The data sources 121 may also include public health databases to obtain information related to disease outbreaks, allergen levels, or other health advisories relevant to the user's geographic location. The data sources 121 may further include electronic health records (EHRs), user's medical report, and personal health history data, subject to user consent and privacy protections. The health data may include information about pre-existing medical conditions, prior diagnoses, medication history, allergies, or mental health assessments, which may facilitate in tailoring recommendations specific to the user's physical and mental health needs. The data sources 121 may also include a nutrition database, local directories of healthy restaurants, and/or databases of social or community events that may align with the user's interests and well-being goals. It should be understood that the data sources 121 may include any other databases that provide relevant information pertaining to the users.

In one instance, data sources 121 may serve as an independent data repository for storing user-related information, and may allow the system 100 to track and manage the user's personal behavioral data, health habits, and/or lifestyle improvements without directly sharing sensitive information with an insurance provider or any service providers. The system 100 may continuously analyze the user's data in the data sources 121 to assess whether they have adopted beneficial behaviors, such as improved physical activity, healthier eating habits, or reduced risky behaviors, that may qualify for cost-saving benefits. Upon determining that the user has met predefined thresholds for positive habit changes, the system 100 may generate a prompt in user device 101 to notify the user of potential financial benefits, such as lower premiums or policy discounts. The user may retain full control over whether to share this data with an insurance provider, ensuring transparency and privacy while still allowing them to leverage their behavioral improvements for monetary advantages. This may foster trust by decoupling personal data storage from direct insurer access, while also enabling informed decision-making regarding data sharing.

In one instance, the database 123 may be any type of database, such as relational, hierarchical, object-oriented, and/or the like, wherein data are organized in any suitable manner, including data tables or lookup tables. In one instance, the database 123 may store content associated with the users (e.g., policyholders), the user device 101, and the analysis platform 103, and may manage multiple types of information that aid in the content provisioning and sharing process. For example, the database 123 may store raw user data collected from the user device 101, sensors, and/or external data sources. Additionally, the database 123 may store processed data, such as categorized data indicative of the user's physical health, mental well-being, and behavioral patterns, as well as user profiles, historical trends, and adherence records.

In another instance, the database 123 may include a machine-learning based training database with a pre-defined mapping defining a relationship between various input parameters and output parameters based upon various statistical methods. For example, the training database may include machine-learning algorithms to learn mappings between input parameters related to the users. In one example, the training database may include a dataset that includes data collections that are not subject-specific, e.g., data collections based upon population-wide observations, local, regional, or super-regional observations, and the like. The training database may be routinely updated and/or supplemented based upon the machine-learning methods.

The various elements of the computing system 100 may communicate with each other through a communication network. In one instance, the user device 101 may include a network detection sensor for detecting wireless signals or receivers for different communications (e.g., Bluetooth, Wi-Fi, Li-Fi, near field communication (NFC), etc.) from the communication network. The communication network may support a variety of different communication protocols and communication techniques.

In one instance, the communication network may allow the user device 101 (or one or more user devices) to communicate with the analysis platform 103. The communication network may include one or more networks such as a data network, a wireless network, a telephony network, or any combination thereof. It is contemplated that the data network is any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network is, for example, a cellular communication network and employs various technologies including 5G (5th Generation), 4G, 3G, 2G, Long Term Evolution (LTE), wireless fidelity (Wi-Fi), Bluetooth®, Internet Protocol (IP) data casting, satellite, mobile ad-hoc network (MANET), vehicle controller area network (CAN bus), and the like, or any combination thereof.

The above presented modules and components of the analysis platform 103 may be implemented in hardware, firmware, software, or a combination thereof. Though depicted as a separate entity in FIG. 1, it is contemplated that the analysis platform 103 may be implemented for direct operation by the respective user device 101. As such, the analysis platform 103 may generate direct signal inputs by way of the operating system of the user device 101. In one instance, one or more of the modules 105-119 may be implemented for operation by the respective user device 101, as the analysis platform 103. The various executions presented herein contemplate any and all arrangements and models.

### Exemplary User Compliance Monitoring and Recommendation Adaptation Flowchart

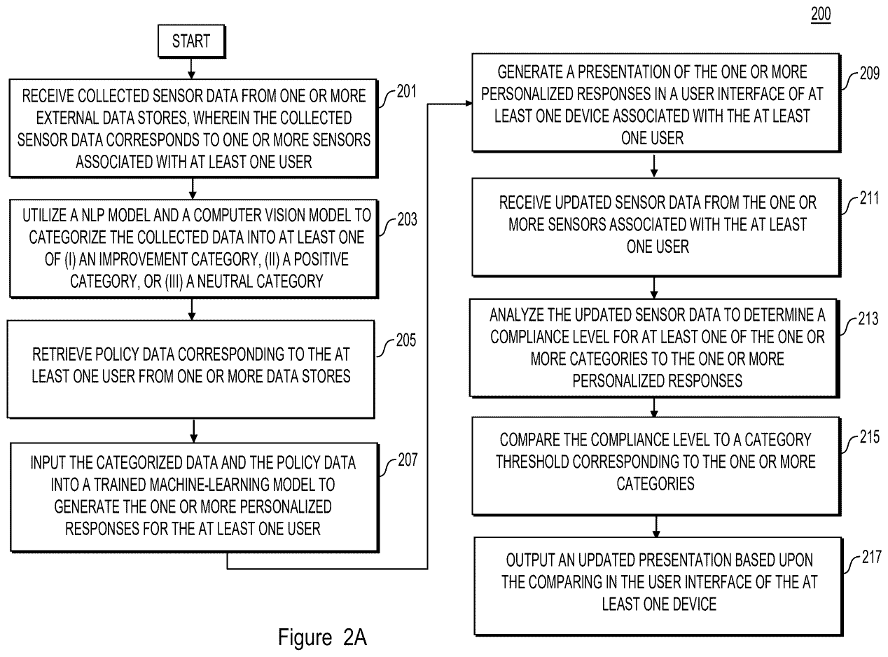

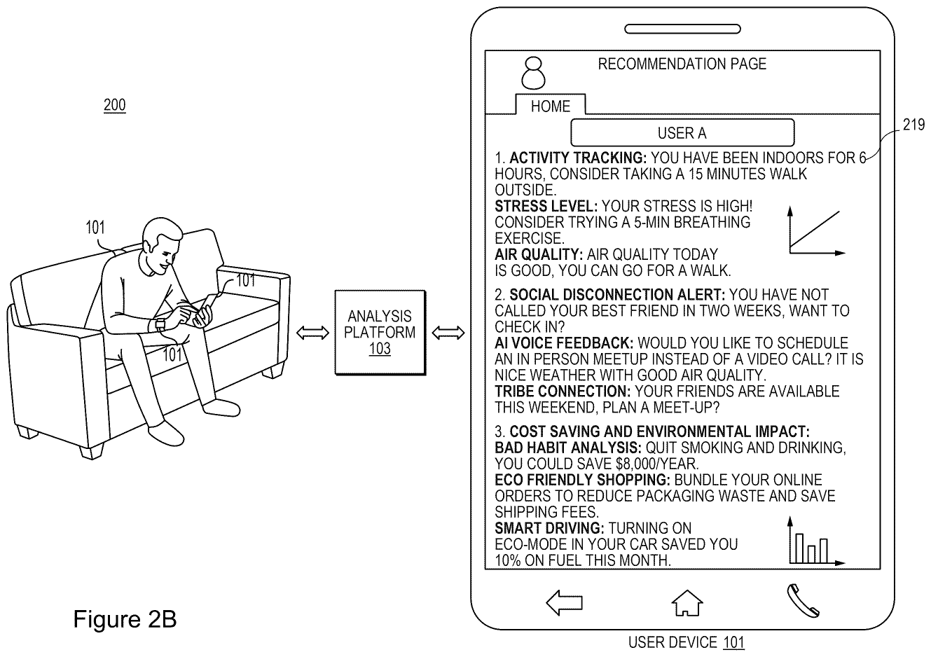

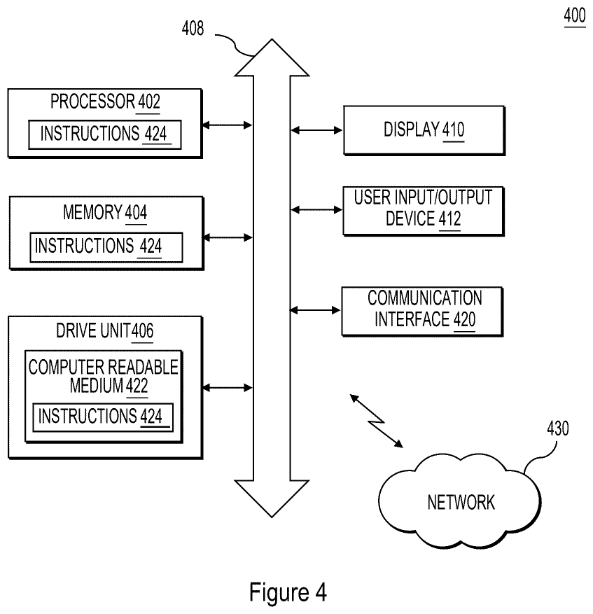

FIG. 2A is an exemplary flowchart of a computer-implemented or computer-based process for analyzing data to generate one or more personalized responses. In one instance, the analysis platform 103 and/or any of the modules 105-119 may perform one or more portions of the process 200 and are implemented using, for instance, a chip set including a processor (e.g., processor 402) and a memory (e.g., memory 404), as shown in FIG. 4. As such, the analysis platform 103 and/or any of the modules 105-119 may be configured to facilitate accomplishing various parts of the process 200, as well as accomplishing embodiments of other processes described herein in conjunction with other components of the computing system 100. Although the process 200 is illustrated and described as a sequence of actions, operations, and/or functionality, it is contemplated that various embodiments of the process 200 may be performed in any order or combination and need not include all of the illustrated actions, operations, and/or functionality.

In block 201, the analysis platform 103 may receive collected sensor data from one or more external data stores (e.g., the data sources 121). The collected sensor data may correspond to one or more sensors associated with a user (e.g., policyholders). In one instance, the sensor data associated with the user may include activity data (e.g., step count, movement patterns, time spent indoors vs. outdoors, etc.), one or more behavioral patterns (e.g., screen time usage, sleep cycles, daily routines), physical health data (e.g., heart rate, blood pressure, body temperature, etc.), mental health data (e.g., stress level indicators, voice tone analysis for the emotional state, self-reported emotional states, mood variations inferred from speech patterns, etc.), one or more social engagement patterns (e.g., frequency of in-person interactions, phone call and message activity, participation in social event, communication event logs, etc.), and/or environmental exposure data (e.g., air quality indices, ambient noise level, ambient temperature, ultraviolet light exposure, etc.). In one example, the data sources 121 may allow users to store and manage their personal data independently, ensuring privacy while still enabling an analysis for potential benefits. The analysis platform 103 may retrieve this information through secure data access protocols, such as API integration, encrypted data transfers, or user-authorized synchronization, ensuring the integrity and confidentiality of the collected data.

In block 203, the analysis platform 103 may utilize an NLP model and a computer vision model to categorize the collected sensor data into one or more categories. In one example, the NLP model may process textual and/or audio-based inputs, such as analyzing speech patterns for stress or mood indicators, and may interpret user-generated queries. In one example, the computer vision model may analyze images and/or video data, such as detecting posture for physical activity assessment or recognizing facial expressions indicative of emotional well-being. By categorizing sensor data, ranging from activity level, behavioral trends, and physical health indicators to environmental conditions and social engagement patterns, the system may generate meaningful insights tailored to the user's well-being.

In one instance, the one or more categories may include an improvement category, a positive category, or a neutral category. In one instance, the improvement category may refer to a categorization of data that identifies areas needing improvement (e.g., positive adjustment) to enhance the overall well-being of the user, such as potential health risks, environmental hazards, social disengagements, financial inefficiencies, and/or stress-related indicators. In one instance, the positive category may refer to a categorization of data that indicates healthy behaviors, favorable environmental conditions, social engagements, and/or financial savings that may contribute to the overall well-being of the user. In one instance, the neutral category may reflect stable conditions that do not need immediate intervention, but are monitored over time to determine whether they transition into the positive or improvement category.

In block 205, the analysis platform 103 may retrieve policy data corresponding to the user from one or more data stores (e.g., data sources 121). In one instance, the policy data may include policy terms (e.g., duration of coverage, renewal conditions, etc.), coverage details (e.g., scope of benefits, exclusions, coverage limits, etc.), eligibility criteria (e.g., qualifying conditions, age or health requirements), claim history (e.g., past claims filed, claim approval rates, claim frequency), risk assessment (e.g., user's lifestyle risk profile, health risk evaluation), and/or premium adjustment factors (e.g., behavior-based discounts, wellness incentives, risk-based premium modification, etc.).

In block 207, the analysis platform 103 may input the categorized data and the policy data into a trained machine-learning model to generate one or more personalized responses (i.e., recommendations) for the user. In one instance, the trained machine-learning model may utilize pattern recognition and/or predictive analysis to assess trends, deviations, and/or correlations within the input data. By analyzing user-specific features, behavioral trends, and policy parameters, the trained machine-learning model may identify one or more actionable insights. The trained machine-learning model may continuously refine its outputs by weighting various input factors and adjusting its decision-making parameters based upon learned patterns from historical and real-time data.

In one instance, the trained machine-learning model may generate recommendations for corrective actions for mitigating identified or predicted risks based upon the data categorized into the improvement category. The one or more corrective actions may include activity suggestion (e.g., recommend going for a walk, spending time outdoors, etc.), personalized health plans (e.g., suggest balanced diet, workout routines, etc.), stress management techniques (e.g., mindfulness exercise, relaxation methods to reduce stress, etc.), community engagement activities (e.g., suggest social events or group activities, etc.), therapy suggestions (e.g., recommend therapy sessions, counseling, other self-help resources, etc.), sleep optimization strategies (e.g., suggest sleep schedules, relaxation techniques for better sleep, etc.), substance use reduction plans (e.g., suggest gradual reduction techniques for smoking or alcohol consumption, suggest support groups, etc.), financial planning strategies (e.g., provide budgeting advice, tips for reducing expenses, etc.), and/or preventive healthcare screenings (e.g., recommend scheduling regular health check-ups or screening based upon age or medical history).

In another instance, the trained machine-learning model may generate and output one or more notifications to the user device 101 based upon the sensor data categorized into the positive category. The notifications may encourage the user by promoting positive activities, offering rewards, delivering personalized motivational messages, awarding achievement-based badges, suggesting similar habits, and/or recommending goal-setting challenges.

In another instance, the trained machine-learning model may generate one or more recommendations to prevent regression into the improvement category or to encourage gradual improvement toward the positive category. The recommendations may include health-conscious behavioral nudges (e.g., reminders to maintain hydration, posture correction alerts, encouraging screen breaks, etc.), social engagement reinforcements (e.g., prompts to check in with friends, join community events, etc.), financial optimization advice (e.g., suggestions for cost savings, etc.), environmental awareness (e.g., notification about sustainable living practices, local air quality, local water quality, local noise level, etc.), mental well-being advice (e.g., mindfulness exercise, etc.), nutritional guidance (e.g., meal planning tips, balanced diet suggestions, etc.), productivity balance (e.g., work-life balance reminders, focus boosting techniques, etc.), and/or physical activity encouragement (e.g., movement challenges, personalized exercise plans, etc.).

In block 209, the analysis platform 103 may generate a presentation of personalized responses in a user interface of a device (e.g., user device 101) associated with the user. In one instance, the presentation may include an interactive visual (e.g., dynamic charts, progress trackers, animated avatars demonstrating exercises, etc.), auditory element (e.g., voice-guided recommendations, notification sounds for reminders, etc.), and/or haptic feedback elements (e.g., gentle vibrations for activity reminders, tactile responses for confirmation actions, etc.).

In block 211, the analysis platform 103 may receive updated sensor data from one or more sensors associated with the user. The updated sensor data may allow the analysis platform 103 to monitor the user's real-time responses and adaptations to previous recommendations, enabling a dynamic feedback loop for personalized interventions. In one instance, the updated sensor data may include motion data (e.g., step count, posture detection, or activity recognition, such as walking, running, sedentary behavior, etc.), biometric signals (e.g., heart rate variability, skin temperature, oxygen saturation levels, stress indicators, etc.), and/or interaction logs (e.g., frequency and duration of interaction with the user interface of the user device 101, acknowledgment of recommendations, or response patterns to personalized interventions, etc.).

In one instance, the analysis platform 103 may analyze motion data to detect patterns, biometric signals to identify physiological responses, and/or the interaction logs to determine user engagement with system prompts. By analyzing motion data, biometric signals, and/or interaction logs, the analysis platform 103 may determine whether the user is actively following recommendations or if further adjustments are needed. For example, if a user was advised to engage in physical activity but the motion data indicates prolonged inactivity, the analysis platform 103 may infer non-adherence and refine future suggestions accordingly. Similarly, biometric signals, such as increased heart rate variability or elevated stress markers may indicate whether the relaxation techniques or stress management recommendations were effective. Additionally, interaction logs may help the analysis platform 103 to assess user engagement with the interface, such as whether they are actively viewing suggestions, dismissing them, or modifying their preferences.

The analysis platform 103 may compare motion data, biometric signals, and/or interaction logs to predefined adherence criteria. The predefined adherence criteria may correspond to expected behaviors or actions associated with the personalized responses (i.e., personalized recommendations), serving as benchmarks for assessing progress. For example, if a recommendation suggests a 30-minute daily walk, the analysis platform 103 may check whether the motion data reflects sustained movement over the expected duration. Similarly, if a stress management technique is recommended, the analysis platform 103 may analyze biometric signals, such as heart rate variability, to determine whether the user's physiological response aligns with the expected relaxation outcome. Interaction logs may further indicate engagement, such as whether the user acknowledged the recommendations, adjusted their routine, or ignored the suggestions. By comparing the updated sensor data with these predefined adherence criteria, the analysis platform 103 may identify deviations, measure the effectiveness of recommendations, and refine future guidance accordingly.

In block 213, the analysis platform 103 may analyze the updated sensor data to determine a compliance level for at least one of the categories to the personalized responses. This compliance level may be assessed based upon how closely the user's behavior, physiological responses, and interactions align with the expected adherence criteria. For instance, if the recommendation involves increased physical activity, the analysis platform 103 may evaluate motion data to quantify whether the user met the suggested step count or activity duration. Similarly, for stress management techniques, the analysis platform 103 may analyze biometric signals to determine whether the stress indicators have decreased over time. The compliance level may be represented as a numerical score, percentage, or qualitative categorization (e.g., high, moderate, low) to provide insights into the effectiveness of the recommendations.

In block 215, the analysis platform 103 may compare the compliance level to a category threshold corresponding to the categories. The category threshold may represent a benchmark or minimum level of adherence required for the recommendation to be considered effective. For example, if a recommendation involves increasing the daily step count, the category threshold may specify that at least 80% of the target step counts be achieved over a given period. Similarly, for stress reduction techniques, the threshold may require a measurable decrease in heart rate variability fluctuations over time. By performing this comparison, the analysis platform 103 may determine whether the user has successfully adopted the recommended actions or if further interventions, modifications, or reinforcements may be necessary. If the compliance level falls below the category threshold, the analysis platform 103 may trigger adaptive strategies, such as personalized reminders or alternative recommendations, to encourage improved adherence.

The analysis platform 103 may retrain the trained machine-learning model based upon the comparison. The updated sensor data and the compliance level may be input as training data into the trained machine-learning model to update one or more model parameters. This retraining process may allow the trained machine-learning model to dynamically adjust one or more model parameters, improving its ability to generate personalized and effective recommendations. For example, if a user consistently fails to adhere to physical activity recommendations, the trained machine-learning model may adjust its parameters to suggest alternative activities that better align with the user's preferences and physical capabilities. Similarly, if the compliance level indicates a strong response to certain stress management techniques, the trained machine-learning model may increase the weighting of those strategies in future recommendations. This iterative learning process may enhance the personalization of recommendations, ensuring that they remain relevant, achievable, and effective in promoting positive behavioral changes.

In block 217, the analysis platform 103 may output an updated presentation based upon the comparison, in the user interface of the device associated with the user. This presentation may incorporate interactive visuals (e.g., progress chart, adherence scores), auditory elements (e.g., spoken feedback, motivational prompts), and/or haptic feedback (e.g., vibration cues for reminders). For instance, if the analysis platform 103 determines that the user has successfully adhered to a recommended activity, the interface may display a congratulatory message along with progress insights. Conversely, if adherence is low, the interface may provide adaptive recommendations, such as adjusting goals or offering alternative strategies, to better align with the user's habits and preferences.

### Exemplary Personalized Recommendations Based Upon Multi-Sensor Data

FIG. 2B illustrates an exemplary scenario in which a user operates within a multi-sensor environment, wherein various sensor-equipped devices continuously transmit real-time physiological, behavioral, and/or contextual data to the analysis platform 103 to receive personalized recommendations, according to one or more embodiments.

In the illustrated example, the user is sitting upon a sofa, engaging with the user device 101 (e.g., smartphone, smartwatch, smart clothing) that may transmit data to the analysis platform 103 for real-time assessment. In one example, the smartwatch may continuously monitor physiological and movement-related data associated with the user, such as heart rate, heart rate variability, step count, physical activity levels, and/or stress indicators derived from biometric signals. The data may be transmitted to the analysis platform 103 to assess the user's physical activity patterns, stress levels, and/or overall well-being. In another example, smart clothing may include wearable textile-integrated sensors, such as smart shirts that may measure respiratory rate, body posture, and/or muscle activity, and smart socks that may monitor gait and/or movement patterns. The data collected from these sensors may be transmitted to the analysis platform 103 to assess sedentary behavior, posture irregularities, and/or physiological stress response. In a further example, the smartphone may collect contextual and behavioral data, such as screen time duration, application usage patterns, social engagement frequency (e.g., call logs, messaging activity, and social media interactions), and/or GPS-based location tracking. The data may be transmitted to the analysis platform 103 to infer social connectivity, screen dependency, and/or indoor/outdoor activity trends. Although the present example depicts a smartwatch, smart clothing, and a smartphone as sources of user data, various other sensors and connected devices may contribute to the data collection process, including smart home devices, ambient air quality sensors, voice assistants, and/or fitness trackers. These sensors may collectively provide a comprehensive dataset to the analysis platform 103 for analysis.

The analysis platform 103 may serve as the central processing unit that may collect vast amounts of data from various sensors and smart devices. Upon receiving the data, the analysis platform 103 may process and normalize the collected data to ensure consistency and accuracy. The analysis platform 103 may categorize the processed data into predefined categories. Once the data is structured and categorized, the analysis platform 103 may utilize machine-learning models to generate personalized recommendations tailored to the user's needs. The machine-learning models may analyze correlations between various data points, predict potential risks, and provide proactive suggestions for lifestyle improvements. The various functions of the analysis platform 103, including data collection, data processing, categorization, utilizing machine-learning models, and/or generating recommendations are described in FIG. 1, and are not elaborated upon here.

The analysis platform 103 may generate a presentation of the personalized recommendations in a user device 101 (e.g., display 219). In one instance, an activity tracking recommendation may be generated based upon real-time sensor data and contextual analysis of the user's behavior. The recommendations may include:
(i) You have been indoors for 6 hours, consider taking a 15-minute walk outside: This recommendation may be based upon data from the user's smartphone (indoor GPS location, screentime) and wearable devices (lack of movement from a smartwatch or smart clothing). The analysis platform 103 may detect prolonged indoor inactivity and may suggest a short walk to encourage physical movement.
(ii) Your stress level is high, consider trying a 5-minute breathing exercise: The smartwatch or smart clothing may monitor heart rate variability (HRV) and other physiological signals indicative of stress. If the detected stress levels exceed a threshold, the analysis platform 103 may recommend a quick breathing exercise to help the user relax and regain focus.
(iii) The air quality today is good, you can go for a walk: The analysis platform 103 may pull data from external environmental databases (e.g., data sources 121). If the air quality index (AQI) is favorable, the analysis platform 103 may suggest an outdoor activity, reinforcing healthy habits when conditions are optimal.

In one instance, a social disconnect notification may be generated to help users maintain meaningful social connections by identifying patterns of social disengagement and providing timely reminders or suggestions. The notifications may include:
(i) You have not called your best friend in two weeks, want to check-in?: This alert may be triggered when the analysis platform 103 detects a decline in communication frequency with a specific close contact, such as a best friend. The analysis platform 103 may prompt the user to reinitiate contact, fostering social bonds and preventing prolonged isolation.
(ii) AI voice feedback: would you like to schedule an in-person meetup instead of a video call? It is nice weather with good air quality: This notification may combine social engagement insights with environmental data. If the system detects a pattern of frequent virtual communication but infrequent in-person interactions, the analysis platform 103 may suggest an alternative. By factoring in weather conditions and air quality reports, the AI may encourage outdoor social interactions when conditions are favorable.
(iii) Tribe connections: your friends are available this weekend, plan a meet-up?: The analysis platform 103 may analyze shared calendars, location data, and previous meetup patterns to determine when friends are likely to be available. By identifying common free time slots, the analysis platform 103 may proactively suggest scheduling a group gathering, helping users maintain their social circles and fostering community engagement.

In one instance, cost-saving and environmental impact recommendations may be generated to help users manage their finances and be considerate of the environment. The recommendations may include:
(i) Quitting smoking and drinking could save you $8,000/year: This recommendation may focus upon providing the user with insights into the potential financial savings associated with quitting certain habits, such as smoking and drinking. The analysis platform 103 may calculate the average annual spending on cigarettes, alcohol, or similar products, factoring in variables such as frequency of use and product cost. The recommendation may highlight how eliminating these habits could lead to significant savings, encourage healthier behaviors, and foster financial awareness.
(ii) Bundle your online orders to reduce packaging waste and save on shipping fees: This recommendation may encourage the user to adopt a more environmentally conscious approach to online shopping by suggesting they bundle their orders. By consolidating multiple items into fewer deliveries, the user may reduce the environmental impact associated with packaging waste, including plastic, cardboard, and other materials. Additionally, bundling items may lead to fewer deliveries, thereby decreasing the carbon footprint from transportation emissions.
(iii) Smart driving: turning on eco-mode in your car saved you 10% of fuel this month: This recommendation may promote the user's adoption of eco-friendly driving habits, specifically by activating their car's ‘eco-mode’ feature. Many modern vehicles come equipped with driving modes designed to optimize fuel efficiency. When the user engages eco-mode, the vehicle adjusts parameters like engine power, acceleration, and/or air conditioning to minimize fuel consumption. The analysis platform 103 may track the user's driving behavior and may calculate fuel savings achieved through the use of eco-mode.

### Exemplary Machine-Learning Techniques

One or more implementations disclosed herein may include and/or may be implemented using a machine-learning model. For example, one or more of the modules of the analysis platform 103 may be implemented using a machine-learning model and/or may be used to train the machine-learning model. A given machine-learning model may be trained using the data flow 300 of FIG. 3. Training data 312 may include one or more of stage inputs 314 and known outcomes 318 related to the machine-learning model to be trained. The stage inputs 314 may be from any applicable source including text, visual representations, data, values, comparisons, stage outputs, e.g., one or more outputs from one or more actions or operations from FIGS. 2A-2B. The known outcomes 318 may be included for the machine-learning models generated based upon supervised or semi-supervised training. An unsupervised machine-learning model may not be trained using known outcomes 318. Known outcomes 318 may include known or desired outputs for future inputs similar to, or in the same category as, stage inputs 314 that do not have corresponding known outputs.

The training data 312 and a training algorithm 320, e.g., one or more of the modules implemented using the machine-learning model and/or may be used to train the machine-learning model, may be provided to a training component 330 that may apply the training data 312 to the training algorithm 320 to generate the machine-learning model. According to an implementation, the training component 330 may be provided comparison results 316 that compare a previous output of the corresponding machine-learning model to apply the previous result to re-train the machine-learning model. The comparison results 316 may be used by training component 330 to update the corresponding machine-learning model. The training algorithm 320 may utilize machine-learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, classifiers such as K-Nearest Neighbors, and/or discriminative models such as Decision Forests and maximum margin methods, models specifically discussed in the present disclosure, or the like.

The machine-learning model used herein may be trained and/or used by adjusting one or more weights and/or one or more layers of the machine-learning model. For example, during training, a given weight may be adjusted (e.g., increased, decreased, removed) based upon training data or input data. Similarly, a layer may be updated, added, or removed based upon training data/and or input data. The resulting outputs may be adjusted based upon the adjusted weights and/or layers.

In general, any process or operation discussed in this disclosure is understood to be computer-implementable, such as the processes illustrated in FIGS. 1-2B may be performed by one or more processors of a computer system as described herein. A process or process action or operation performed by one or more processors may also be referred to as an operation. The one or more processors may be configured to perform such processes by having access to instructions (e.g., software or computer-readable code) that, when executed by one or more processors, cause the one or more processors to perform the processes. The instructions may be stored in a memory of the computer system. A processor may be a central processing unit (CPU), a graphics processing unit (GPU), or any suitable type of processing unit.

A computer system, such as a system or device implementing a process or operation in the examples above, may include one or more computing devices. One or more processors of a computer system may be included in a single computing device or distributed among a plurality of computing devices. One or more processors of a computer system may be connected to a data storage device. A memory of the computer system may include the respective memory of each computing device of the plurality of computing devices.

### Exemplary Computing Environment

In general, any process or operation discussed in this disclosure is understood to be computer-implementable, such as the processes illustrated in FIGS. 1-2B and may be performed by one or more processors of a computer system as described herein. A process or process action or operation performed by one or more processors may also be referred to as an operation. The one or more processors may be configured to perform such processes by having access to instructions (e.g., software or computer-readable code) that, when executed by one or more processors, cause the one or more processors to perform the processes. The instructions may be stored in a memory of the computer system. A processor may be a central processing unit (CPU), a graphics processing unit (GPU), or any suitable type of processing unit.

A computer system, such as a system or device implementing a process or operation in the examples above, may include one or more computing devices. One or more processors of a computer system may be included in a single computing device or distributed among a plurality of computing devices. One or more processors of a computer system may be connected to a data storage device. A memory of the computer system may include the respective memory of each computing device of the plurality of computing devices.

FIG. 4 illustrates an implementation of a computer system that may execute techniques presented herein. The computer system 400 may include a set of instructions that can be executed to cause the computer system 400 to perform any one or more of the methods or computer based functions disclosed herein. The computer system 400 may operate as a standalone device or may be connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, “analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data, e.g., from registers and/or memory to transform that electronic data into other electronic data that, e.g., may be stored in registers and/or memory. A “computer,” a “computing machine,” a “computing platform,” a “computing device,” or a “server” may include one or more processors.

In a networked deployment, the computer system 400 may operate in the capacity of a server or as a client user computer in a server-client user network environment, or as a peer computer system in a peer-to-peer (or distributed) network environment. The computer system 400 can also be implemented as or incorporated into various devices, such as a personal computer (PC), a tablet PC, a Personal Digital Assistant (PDA), a mobile device, a palmtop computer, a laptop computer, a desktop computer, a web appliance, or any other machine capable of executing a set of instructions (sequential or otherwise) that specify actions to be taken by that machine. In a particular implementation, the computer system 400 may be implemented using electronic devices that provide voice, video, or data communication. Further, while the computer system 400 is illustrated as a single system, the term “system” shall also be taken to include any collection of systems or sub-systems that individually or jointly execute a set, or multiple sets, of instructions to perform one or more computer functions.

As illustrated in FIG. 4, the computer system 400 may include a processor 402, e.g., a Central Processing Unit (CPU), a Graphics Processing Unit (GPU), or both. The processor 402 may be a component in a variety of systems. For example, the processor 402 may be part of a standard personal computer or a workstation. The processor 402 may be one or more processors, digital signal processors, application specific integrated circuits, field programmable gate arrays, servers, networks, digital circuits, analog circuits, combinations thereof, or other now known or later developed devices for analyzing and processing data. The processor 402 may implement a software program, such as code generated manually (i.e., programmed).

The computer system 400 may include a memory 404 that can communicate via bus 408. The memory 404 may be a main memory, a static memory, or a dynamic memory. The memory 404 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 404 may include a cache or random-access memory for the processor 402. In alternative implementations, the memory 404 is separate from the processor 402, such as a cache memory of a processor, the system memory, or other memory.

The memory 404 may be an external storage device or database for storing data. Examples may include a hard drive, compact disc (“CD”), digital video disc (“DVD”), memory card, memory stick, floppy disc, universal serial bus (“USB”) memory device, or any other device operative to store data. The memory 404 is operable to store instructions executable by the processor 402. The functions, acts or tasks illustrated in the figures or described herein may be performed by the processor 402 executing the instructions stored in the memory 404. The functions, acts, or tasks are independent of the particular type of instruction set, storage media, processor or processing strategy and may be performed by software, hardware, integrated circuits, firmware, micro-code and the like, operating alone or in combination. Likewise, processing strategies may include multiprocessing, multitasking, parallel processing, and the like.

As shown, the computer system 400 may further include a display 410, such as a liquid crystal display (LCD), an organic light emitting diode (OLED), a flat panel display, a solid-state display, a cathode ray tube (CRT), a projector, a printer or other now known or later developed display device for outputting determined information. The display 410 may act as an interface for the user to see the functioning of the processor 402, or specifically as an interface with the software stored in the memory 404 or in the drive unit 406.

Additionally or alternatively, the computer system 400 may include an input/output device 412 configured to allow a user to interact with any of the components of the computer system 400. The input/output device 412 may be a number pad, a keyboard, or a cursor control device, such as a mouse, or a joystick, touch screen display, remote control, or any other device operative to interact with the computer system 400.

The computer system 400 may also or alternatively include drive unit 406 implemented as a disk or optical drive. The drive unit 406 may include a computer-readable medium 422 in which one or more sets of instructions 424, e.g., software, can be embedded. Further, instructions 424 may embody one or more of the methods or logic as described herein. The instructions 424 may reside completely or partially within the memory 404 and/or within the processor 402 during execution by the computer system 400. The memory 404 and the processor 402 also may include computer-readable media as discussed above.

In some systems, computer-readable medium 422 may include the set of instructions 424 or receive and execute the set of instructions 424 responsive to a propagated signal so that a device connected to network 430 can communicate voice, video, audio, images, or any other data over the network 430. Further, the set of instructions 424 may be transmitted or received over the network 430 via communication port or interface 420, and/or using bus 408. The communication port or interface 420 may be a part of the processor 402 or may be a separate component. The communication port or interface 420 may be created in software or may be a physical connection in hardware.

The communication port or interface 420 may be configured to connect with a network 430, external media, the display 410, or any other components in computer system 400, or combinations thereof. The connection with the network 430 may be a physical connection, such as a wired Ethernet connection or may be established wirelessly as discussed below. Likewise, the additional connections with other components of the computer system 400 may be physical connections or may be established wirelessly. The network 430 may alternatively be directly connected to the bus 408.

While the computer-readable medium 422 is shown to be a single medium, the term “computer-readable medium” may include a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that causes a computer system to perform any one or more of the methods or operations disclosed herein. The computer-readable medium 422 may be non-transitory, and may be tangible.

The computer-readable medium 422 may include a solid-state memory such as a memory card or other package that houses one or more non-volatile read-only memories. The computer-readable medium 422 can be a random-access memory or other volatile re-writable memory. Additionally or alternatively, the computer-readable medium 422 may include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, can be constructed to implement one or more of the methods described herein. Applications that may include the apparatus and systems of various implementations may broadly include a variety of electronic and computer systems. One or more implementations described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

Computer system 400 may be connected to network 430. The network 430 may define one or more networks including wired or wireless networks. The wireless network may be a cellular telephone network, an 802.10, 802.16, 802.20, or WiMAX network. Further, such networks may include a public network, such as the Internet, a private network, such as an intranet, or combinations thereof, and may utilize a variety of networking protocols now available or later developed including, but not limited to TCP/IP based networking protocols. The network 430 may include wide area networks (WAN), such as the Internet, local area networks (LAN), campus area networks, metropolitan area networks, a direct connection such as through a Universal Serial Bus (USB) port, or any other networks that may allow for data communication.

The network 430 may be configured to couple one computing device to another computing device to enable communication of data between the devices. The network 430 may generally be enabled to employ any form of machine-readable media for communicating information from one device to another. The network 430 may include communication methods by which information may travel between computing devices.

The network 430 may be divided into sub-networks. The sub-networks may allow access to all of the other components connected thereto or the sub-networks may restrict access between the components. The network 430 may be regarded as a public or private network connection and may include, for example, a virtual private network or an encryption or other security mechanism employed over the public Internet, or the like.

### Exemplary Embodiment

A computer-implemented method for analyzing data to generate one or more personalized responses may be provided. The computer-implemented method may be performed by one or more local or remote processors of a computing system in communication with one or more local or remote data sources. The computer-implemented method may include (1) receiving, by the one or more processors, collected sensor data from one or more external data stores, wherein the collected sensor data corresponds to one or more sensors associated with at least one user; (2) utilizing, by the one or more processors, a natural language processing (NLP) model and a computer vision model to categorize the collected sensor data into one or more categories, wherein the one or more categories include at least one of (i) an improvement category, (ii) a positive category, or (iii) a neutral category; (3) retrieving, by the one or more processors, policy data corresponding to the at least one user from one or more data stores; (4) inputting, by the one or more processors, the categorized data and the policy data into a trained machine-learning model to generate the one or more personalized responses for the at least one user; (5) generating, by the one or more processors, a presentation of the one or more personalized responses in a user interface of at least one device associated with the at least one user; (6) receiving, by the one or more processors, updated sensor data from the one or more sensors of the at least one user; (7) analyzing, by the one or more processors, the updated sensor data to determine a compliance level for at least one of the one or more categories to the one or more personalized responses; (8) comparing, by the one or more processors, the compliance level to a category threshold corresponding to the one or more categories; and/or (9) outputting, by the one or more processors, an updated presentation based upon the comparing in the user interface of the at least one device. The presentations may be visually and/or verbally presented to a user such as via a voice bot, display screen, speaker, and/or other input/output devices.

In some embodiments, the voice bots or chatbots may be configured to utilize AI and/or ML techniques, such as for input or output devices. For instance, a voice bot or chatbot may be a ChatGPT chatbot, an InstructGPT bot, a Codex bot, or a Google Bard bot. The voice bot or chatbot may employ supervised or unsupervised ML techniques, which may be followed by, and/or used in conjunction with, reinforced or reinforcement learning techniques. The voice bot or chatbot may employ the techniques utilized for ChatGPT, InstructGPT bot, Codex bot, or Google Bard bot.

Additionally or alternatively, the data associated with the at least one user may include (i) activity data, (ii) one or more behavioral patterns, (iii) physical health data, (iv) mental health data, (v) one or more social engagement patterns, and/or (vi) environmental exposure data.

In certain aspects, the policy data may include (i) one or more policy terms, (ii) one or more coverage details, (iii) one or more eligibility criteria, (iv) a claim history, (v) risk assessment, and/or (vi) premium adjustment factors.

In various embodiments, generating the one or more personalized responses by the trained machine-learning model may include generating one or more recommendations for one or more corrective actions for mitigating identified or predicted risks based upon the data categorized into the improvement category, wherein the one or more corrective actions may include (i) an activity suggestion, (ii) one or more personalized health plans, (iii) one or more stress management techniques, (iv) one or more community engagement activities, (v) one or more therapy suggestions, (vi) one or more sleep optimization strategies, (vii) one or more substance use reduction plans, (viii) one or more financial planning strategies, and/or (ix) one or more preventive healthcare screenings.

In certain embodiments, generating the one or more personalized responses by the trained machine-learning model may include generating one or more notifications based upon the sensor data categorized into the positive category, wherein the one or more notifications may encourage the at least one user by (i) promoting one or more positive activities, (ii) offer one or more rewards, (iii) deliver one or more personalized motivational messages, (iv) award one or more achievement-based badges, (v) suggest one or more similar habits, and/or (vi) recommend one or more goal-setting challenges.

In some embodiments, the presentation of the one or more personalized responses in the user interface may include (i) an interactive visual, (ii) one or more auditory elements, and/or (iii) one or more haptic feedback elements.

Additionally or alternatively, the updated sensor data may include (i) motion data, (ii) one or more biometric signals, and/or (iii) one or more interaction logs.

In various embodiments, receiving the updated sensor data from the one or more sensors of the at least one user may include (i) analyzing, by the one or more processors, the motion data to detect one or more patterns, the one or more biometric signals to identify one or more physiological responses, or the one or more interaction logs to determine user engagement with one or more system prompts; and/or (ii) comparing, by the one or more processors, the motion data, the one or more biometric signals, or the one or more interaction logs to one or more predefined adherence criteria, wherein the one or more predefined adherence criteria correspond to one or more expected behaviors or one or more actions associated with the one or more personalized responses.

In certain aspects, retraining, by the one or more processors, the trained machine-learning model based upon the comparing.

In some embodiments, retraining the trained machine-learning model may include inputting, by the one or more processors, the updated sensor data and the compliance level as training data into the trained machine-learning model to update one or more model parameters.

A computer system for analyzing data to generate one or more personalized responses may be provided. The computer system may include one or more processors of a computing system, and at least one non-transitory computer readable medium storing instructions which, when executed by the one or more processors, cause the one or more processors to perform operations. The computer system may perform operations including (1) receiving collected sensor data from one or more external data stores, wherein the collected sensor data corresponds to one or more sensors associated with at least one user; (2) utilizing an NLP model and a computer vision model to categorize the collected sensor data into one or more categories, wherein the one or more categories include at least one of (i) an improvement category, (ii) a positive category, or (iii) a neutral category; (3) retrieving policy data corresponding to the at least one user from one or more data stores; (4) inputting the categorized data and the policy data into a trained machine-learning model to generate the one or more personalized responses for the at least one user; (5) generating a presentation of the one or more personalized responses in a user interface of at least one device associated with the at least one user; (6) receiving updated sensor data from the one or more sensors of the at least one user; (7) analyzing the updated sensor data to determine a compliance level for at least one of the one or more categories to the one or more personalized responses; (8) comparing the compliance level to a category threshold corresponding to the one or more categories; and/or (9) outputting an updated presentation based upon the comparing in the user interface of the at least one device.

Additionally or alternatively, the data associated with the at least one user may include (i) activity data, (ii) one or more behavioral patterns, (iii) physical health data, (iv) mental health data, (v) one or more social engagement patterns, and/or (vi) environmental exposure data.

In certain aspects, the policy data may include (i) one or more policy terms, (ii) one or more coverage details, (iii) one or more eligibility criteria, (iv) a claim history, (v) risk assessment, and/or (vi) premium adjustment factors.

In various embodiments, generating the one or more personalized responses by the trained machine-learning model may include generating one or more recommendations for one or more corrective actions for mitigating identified or predicted risks based upon the data categorized into the improvement category, wherein the one or more corrective actions may include (i) an activity suggestion, (ii) one or more personalized health plans, (iii) one or more stress management techniques, (iv) one or more community engagement activities, (v) one or more therapy suggestions, (vi) one or more sleep optimization strategies, (vii) one or more substance use reduction plans, (viii) one or more financial planning strategies, and/or (ix) one or more preventive healthcare screenings.

In certain embodiments, generating the one or more personalized responses by the trained machine-learning model may include generating one or more notifications based upon the sensor data categorized into the positive category, wherein the one or more notifications may encourage the at least one user by (i) promoting one or more positive activities, (ii) offer one or more rewards, (iii) deliver one or more personalized motivational messages, (iv) award one or more achievement-based badges, (v) suggest one or more similar habits, and/or (vi) recommend one or more goal-setting challenges.

In some embodiments, the presentation of the one or more personalized responses in the user interface may include (i) an interactive visual, (ii) one or more auditory elements, and/or (iii) one or more haptic feedback elements.

Additionally or alternatively, the updated sensor data may include (i) motion data, (ii) one or more biometric signals, and/or (iii) one or more interaction logs.

A non-transitory computer readable medium for analyzing data to generate one or more personalized responses may be provided. The non-transitory computer readable medium may store instructions which, when executed by one or more processors, cause the one or more processors to perform operations. The one or more processors may perform operations including (1) receiving collected sensor data from one or more external data stores, wherein the collected sensor data corresponds to one or more sensors associated with at least one user; (2) utilizing an NLP model and a computer vision model to categorize the collected sensor data into one or more categories, wherein the one or more categories include at least one of (i) an improvement category, (ii) a positive category, or (iii) a neutral category; (3) retrieving policy data corresponding to the at least one user from one or more data stores; (4) inputting the categorized data and the policy data into a trained machine-learning model to generate the one or more personalized responses for the at least one user; (5) generating a presentation of the one or more personalized responses in a user interface of at least one device associated with the at least one user; (6) receiving updated sensor data from the one or more sensors of the at least one user; (7) analyzing the updated sensor data to determine a compliance level for at least one of the one or more categories to the one or more personalized responses; (8) comparing the compliance level to a category threshold corresponding to the one or more categories; and/or (9) outputting an updated presentation based upon the comparing in the user interface of the at least one device.

Additionally or alternatively, the data associated with the at least one user may include (i) activity data, (ii) one or more behavioral patterns, (iii) physical health data, (iv) mental health data, (v) one or more social engagement patterns, and/or (vi) environmental exposure data.

In certain aspects, the policy data may include (i) one or more policy terms, (ii) one or more coverage details, (iii) one or more eligibility criteria, (iv) a claim history, (v) risk assessment, and/or (vi) premium adjustment factors.

### Additional Considerations

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the actions, operations, and/or functionality of computer-implemented methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure may be implemented using any appropriate techniques for implementing the functionality described herein. The disclosure is not limited to any particular programming language or operating system.

The present embodiments envision collecting and analyzing user or customer data, such as sensor data, mobile device data, smart or autonomous data, smart appliance data, etc. associated with the user or customer devices or computing devices. Such data may be collected and analyzed once the user or customer affirmatively opts into a program, such as a customer loyalty or rewards program, or the customer otherwise affirmatively consents to data being generated by their devices being utilized, collected, and/or analyzed.

Although the text herein sets forth a detailed description of numerous different embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘___’ is hereby defined to mean . . . ” or a similar sentence, there is no intent to limit the meaning of that term, either expressly or by implication, beyond its plain or ordinary meaning, and such term should not be interpreted to be limited in scope based upon any statement made in any section of this patent (other than the language of the claims). To the extent that any term recited in the claims at the end of this disclosure is referred to in this disclosure in a manner consistent with a single meaning, that is done for sake of clarity only so as to not confuse the reader, and it is not intended that such claim term be limited, by implication or otherwise, to that single meaning.

Finally, unless a claim element is defined by expressly reciting the word “means” and a function without the recital of any structure, it is not intended that the scope of any claim element be interpreted based upon the application of 35 U.S.C. § 112(f).

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in exemplary configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In exemplary embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations). A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate upon a resource (e.g., a collection of information).

The various operations of exemplary methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some exemplary embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of geographic locations.

Unless specifically stated otherwise, discussions herein using words such as processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

Upon reading this disclosure, those of skill in the art will appreciate still additional alternative structural and functional designs for the approaches described herein. Therefore, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The particular features, structures, or characteristics of any specific embodiment may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein.

It is therefore intended that the foregoing detailed description be regarded as illustrative rather than limiting, and that it be understood that it is the following claims, including all equivalents, that are intended to define the spirit and scope of this invention.

1. A computer-implemented method for analyzing data to generate one or more personalized responses, the computer-implemented method performed by one or more processors of a computing system in communication with one or more data sources, the computer-implemented method comprising:
receiving, by the one or more processors, collected sensor data from one or more external data stores, wherein the collected sensor data corresponds to one or more sensors associated with at least one user, and wherein the collected sensor data includes voice data and visual data associated with the at least one user;
utilizing, by the one or more processors, a natural language processing (NLP) model and a computer vision model to categorize the collected sensor data into one or more categories, wherein the one or more categories include at least one of (i) an improvement category, (ii) a positive category, or (iii) a neutral category, wherein the computer vision model analyzes the visual data to detect one or more facial expressions of the at least one user to identify one or more emotional indicators and body posture data that indicate physical comfort or discomfort, and wherein the NLP model analyzes the voice data to detect one or more changes in speech tone and a speech pace of the at least one user that indicate relaxation or stress, and wherein the categorizing is based upon the facial expressions and the one or more changes in speech tone and the speech pace;
retrieving, by the one or more processors, policy data corresponding to the at least one user from one or more data stores;
inputting, by the one or more processors, the categorized data and the policy data into a trained machine-learning model to generate the one or more personalized responses for the at least one user;
generating, by the one or more processors, a presentation of the one or more personalized responses in a user interface of at least one device associated with the at least one user;
receiving, by the one or more processors, updated sensor data from the one or more sensors associated with the at least one user, wherein the updated sensor data includes updated voice data and updated visual data;
analyzing, by the one or more processors, the updated sensor data to determine a compliance level for at least one of the one or more categories to the one or more personalized responses, wherein the analyzing includes processing the updated voice data and the updated visual data to determine adherence by the at least one user to the one or more personalized responses;
comparing, by the one or more processors, the compliance level to a category threshold corresponding to the one or more categories, wherein the compliance level below the category threshold triggers one or more adaptive strategies including one or more alternative recommendations; and
outputting, by the one or more processors, an updated presentation based upon the comparing in the user interface of the at least one device.

2. The computer-implemented method of claim 1, wherein the sensor data associated with the at least one user includes (i) activity data, (ii) one or more behavioral patterns, (iii) physical health data, (iv) mental health data, (v) one or more social engagement patterns, or (vi) environmental exposure data.

3. The computer-implemented method of claim 1, wherein the policy data includes (i) one or more policy terms, (ii) one or more coverage details, (iii) one or more eligibility criteria, (iv) a claim history, (v) risk assessment, or (vi) premium adjustment factors.

4. The computer-implemented method of claim 3, wherein generating the one or more personalized responses by the trained machine-learning model, further comprises:
generating one or more recommendations for one or more corrective actions for mitigating identified or predicted risks based upon the data categorized into the improvement category, wherein the one or more corrective actions include (i) an activity suggestion, (ii) one or more personalized health plans, (iii) one or more stress management techniques, (iv) one or more community engagement activities, (v) one or more therapy suggestions, (vi) one or more sleep optimization strategies, (vii) one or more substance use reduction plans, (viii) one or more financial planning strategies, or (ix) one or more preventive healthcare screenings.

5. The computer-implemented method of claim 3, wherein generating the one or more personalized responses by the trained machine-learning model, further comprises:
generating one or more notifications based upon the sensor data categorized into the positive category, wherein the one or more notifications encourage the at least one user by (i) promoting one or more positive activities, (ii) offer one or more rewards, (iii) deliver one or more personalized motivational messages, (iv) award one or more achievement-based badges, (v) suggest one or more similar habits, or (vi) recommend one or more goal-setting challenges, and wherein the trained machine-learning model identifies an improvement trend in the categorized data and reinforces the improvement trend by providing one or more incentivized goals to maintain a positive trajectory.

6. The computer-implemented method of claim 1, wherein the presentation of the one or more personalized responses in the user interface includes (i) an interactive visual, (ii) one or more auditory elements, or (iii) one or more haptic feedback elements.

7. The computer-implemented method of claim 1, wherein the updated sensor data further includes (i) motion data, (ii) one or more biometric signals, or (iii) one or more interaction logs.

8. The computer-implemented method of claim 7, wherein receiving the updated sensor data from the one or more sensors associated with the at least one user, further comprises:
analyzing, by the one or more processors, the motion data to detect one or more patterns, the one or more biometric signals to identify one or more physiological responses, or the one or more interaction logs to determine user engagement with one or more system prompts; and
comparing, by the one or more processors, the motion data, the one or more biometric signals, or the one or more interaction logs to one or more predefined adherence criteria, wherein the one or more predefined adherence criteria correspond to one or more expected behaviors or one or more actions associated with the one or more personalized responses.

9. The computer-implemented method of claim 8, further comprising:
inputting, by the one or more processors, the updated sensor data and the compliance level as training data into the trained machine-learning model to update one or more model parameters; and
retraining, by the one or more processors, the trained machine-learning model based upon the comparing, wherein the retraining dynamically adjusts the one or more model parameters to increase a weighting of strategies in future recommendations upon determining the compliance level is above the category threshold.

10. (canceled)

11. A computer system for analyzing data to generate one or more personalized responses, comprising:
one or more processors of a computing system in communication with one or more data sources; and
at least one non-transitory computer readable medium storing instructions which, when executed by the one or more processors, cause the one or more processors to perform operations comprising:
receiving collected sensor data from one or more external data stores, wherein the collected sensor data corresponds to one or more sensors associated with at least one user, and wherein the collected sensor data includes voice data and visual data associated with the at least one user;
utilizing a natural language processing (NLP) model and a computer vision model to categorize the collected sensor data into one or more categories, wherein the one or more categories include at least one of (i) an improvement category, (ii) a positive category, or (iii) a neutral category, wherein the computer vision model analyzes the visual data to detect one or more facial expressions of the at least one user to identify one or more emotional indicators and body posture data that indicate physical comfort or discomfort, and wherein the NLP model analyzes the voice data to detect one or more changes in speech tone and a speech pace of the at least one user that indicate relaxation or stress, and wherein the categorizing is based upon the facial expressions and the one or more changes in speech tone and the speech pace;
retrieving policy data corresponding to the at least one user from one or more data stores;
inputting the categorized data and the policy data into a trained machine-learning model to generate the one or more personalized responses for the at least one user;
generating a presentation of the one or more personalized responses in a user interface of at least one device associated with the at least one user;
receiving updated sensor data from the one or more sensors associated with the at least one user, wherein the updated sensor data includes updated voice data and updated visual data;
analyzing the updated sensor data to determine a compliance level for at least one of the one or more categories to the one or more personalized responses, wherein the analyzing includes processing the updated voice data and the updated visual data to determine adherence by the at least one user to the one or more personalized responses;
comparing the compliance level to a category threshold corresponding to the one or more categories, wherein the compliance level below the category threshold triggers one or more adaptive strategies including one or more alternative recommendations; and
outputting an updated presentation based upon the comparing in the user interface of the at least one device.

12. The system of claim 11, wherein the sensor data associated with the at least one user includes (i) activity data, (ii) one or more behavioral patterns, (iii) physical health data, (iv) mental health data, (v) one or more social engagement patterns, or (vi) environmental exposure data.

13. The system of claim 11, wherein the policy data includes (i) one or more policy terms, (ii) one or more coverage details, (iii) one or more eligibility criteria, (iv) a claim history, (v) risk assessment, or (vi) premium adjustment factors.

14. The system of claim 13, wherein generating the one or more personalized responses by the trained machine-learning model, further comprises:
generating one or more recommendations for one or more corrective actions for mitigating identified or predicted risks based upon the data categorized into the improvement category, wherein the one or more corrective actions include (i) an activity suggestion, (ii) one or more personalized health plans, (iii) one or more stress management techniques, (iv) one or more community engagement activities, (v) one or more therapy suggestions, (vi) one or more sleep optimization strategies, (vii) one or more substance use reduction plans, (viii) one or more financial planning strategies, or (ix) one or more preventive healthcare screenings.

15. The system of claim 13, wherein generating the one or more personalized responses by the trained machine-learning model, further comprises:
generating one or more notifications based upon the sensor data categorized into the positive category, wherein the one or more notifications encourage the at least one user by (i) promoting one or more positive activities, (ii) offer one or more rewards, (iii) deliver one or more personalized motivational messages, (iv) award one or more achievement-based badges, (v) suggest one or more similar habits, or (vi) recommend one or more goal-setting challenges, and wherein the trained machine-learning model identifies an improvement trend in the categorized data and reinforces the improvement trend by providing one or more incentivized goals to maintain a positive trajectory.

16. The system of claim 11, wherein the presentation of the one or more personalized responses in the user interface includes (i) an interactive visual, (ii) one or more auditory elements, or (iii) one or more haptic feedback elements.

17. The system of claim 11, wherein the updated sensor data further includes (i) motion data, (ii) one or more biometric signals, or (iii) one or more interaction logs.

18. A non-transitory computer readable medium for analyzing data to generate one or more personalized responses, the non-transitory computer readable medium storing instructions which, when executed by one or more processors of a computing system in communication with one or more data sources, cause the one or more processors to perform operations comprising:
receiving collected sensor data from one or more external data stores, wherein the collected sensor data corresponds to one or more sensors associated with at least one user, and wherein the collected sensor data includes voice data and visual data associated with the at least one user;
utilizing a natural language processing (NLP) model and a computer vision model to categorize the collected sensor data into one or more categories, wherein the one or more categories include at least one of (i) an improvement category, (ii) a positive category, or (iii) a neutral category, wherein the computer vision model analyzes the visual data to detect one or more facial expressions of the at least one user to identify one or more emotional indicators and body posture data that indicate physical comfort or discomfort, and wherein the NLP model analyzes the voice data to detect one or more changes in speech tone and a speech pace of the at least one user that indicate relaxation or stress, and wherein the categorizing is based upon the facial expressions and the one or more changes in speech tone and the speech pace;
retrieving policy data corresponding to the at least one user from one or more data stores;
inputting the categorized data and the policy data into a trained machine-learning model to generate the one or more personalized responses for the at least one user;
generating a presentation of the one or more personalized responses in a user interface of at least one device associated with the at least one user;
receiving updated sensor data from the one or more sensors associated with the at least one user, wherein the updated sensor data includes updated voice data and updated visual data;
analyzing the updated sensor data to determine a compliance level for at least one of the one or more categories to the one or more personalized responses, wherein the analyzing includes processing the updated voice data and the updated visual data to determine adherence by the at least one user to the one or more personalized responses;
comparing the compliance level to a category threshold corresponding to the one or more categories, wherein the compliance level below the category threshold triggers one or more adaptive strategies including one or more alternative recommendations; and
outputting an updated presentation based upon the comparing in the user interface of the at least one device.

19. The non-transitory computer readable medium of claim 18, wherein the sensor data associated with the at least one user includes (i) activity data, (ii) one or more behavioral patterns, (iii) physical health data, (iv) mental health data, (v) one or more social engagement patterns, or (vi) environmental exposure data.

20. The non-transitory computer readable medium of claim 18, wherein the policy data includes (i) one or more policy terms, (ii) one or more coverage details, (iii) one or more eligibility criteria, (iv) a claim history, (v) risk assessment, or (vi) premium adjustment factors.

21. The computer-implemented method of claim 1, further comprising:
detecting, by the one or more processors, a presence of one or more individuals in an environment associated with the at least one user based upon the visual data;
detecting, by the one or more processors, a communication frequency of the at least one user with one or more contacts;
determining, by the one or more processors, a social engagement level of the at least one user is below a threshold based upon the detected presence and the detected communication frequency; and
generating, by the one or more processors, one or more notifications prompting the at least one user to reinitiate contact with the one or more contacts.
